(19) United States
(12) Patent Application Publication
(10) Pub. No.: US 2022/0274917 A1
(43) Pub. Date: Sep. 1, 2022

(54) SOLVENT FREE CONTINUOUS PROCESS FOR THE SYNTHESIS OF METFORMIN HYROCHLORIDE

(71) Applicant: COUNCIL OF SCIENTIFIC AND INDUSTRIAL RESEARCH, New Delhi (IN)

(72) Inventors: Amol Arvind KULKARNI, Maharashtra Pune (IN); Brijesh SHARMA, Maharashtra Pune (IN)

(21) Appl. No.: 17/753,239

(22) PCT Filed: Sep. 4, 2020
(86) PCT No.: PCT/IN2020/050775

§ 371 Date: Feb. 24, 2022

(30) Foreign Application Priority Data
Sep. 6, 2019 (IN) 201911035946

(51) Int. Cl. C07C 277/08

(52) U.S. Cl. CPC C07C 277/08

(57) ABSTRACT

The present invention provides a continuous solvent free and additive free process for the synthesis of Metformin Hydrochloride in a screw reactor. >90% conversion of the solid substrates without solvent is obtained in a screw reactor to produce Metformin Hydrochloride with at least 90% selectivity.

### FIELD OF THE INVENTION

The invention relates to a continuous process for the synthesis of metformin hydrochloride. More particularly, the invention discloses a solvent free continuous process for the synthesis of metformin hydrochloride using screw reactors.

### BACKGROUND AND PRIOR ART OF THE INVENTION

Metformin is one of the most widely used drug globally to treat Type II diabetes. It has been used since the last nearly 60 years and is still the drug of choice for many patients, either alone or in combination with drugs such as rosiglitazone and sitagliptin for the effective clinical management of blood glucose levels. Between 2014 and 2016 over 80 million prescriptions for metformin have been filled annually in US alone, doubling from around 40 million around 2004. This clearly indicates the quantum of metformin bulk drug needed globally.

There is a commendable body of literature in public domain in the form of patents as well scientific publications describing the possible routes of synthesis of metformin Most of this literature dislcose processes for synthesis of metformin in batch mode. Recently, there have been attempts to improve the synthetic processes of metformin to economize the process or to reduce the content of impurities.

CN102516130 discloses a process for synthesis of metformin comprising several steps with multiple variations in reaction conditions and also a fairly lengthy purification procedure.

US20110021634 provides a process to reduce the dimethyl amine content from metformin from 15 ppm to less than 5 ppm by

a) providing metformin hydrochloride having dimethyl amine content more than 15 ppm;
(b) pulverizing the metformin hydrochloride;
(c) slurrying the metformin hydrochloride in one or more C₁-C₄ alcohol solvents; and
(d) isolating the metformin hydrochloride.

So, till recent times there have been attempts to economize the process of synthesizing metformin as well to provide a purer material. But there still exists a dire need in the art to provide simpler and economically more relevant processes for the synthesis of valuable chemicals such as metformin with greater purity.

### OBJECTS OF THE INVENTION

The main objective of the invention to provide a simple, economically viable process for the synthesis of metformin hydrochloride with high degree of purity and almost complete conversion of the substrates.

### SUMMARY OF THE INVENTION

Accordingly, the present invention provides a continuous process for the synthesis of pure chemicals using a solvent free approach employing a screw reactor.

In an aspect of the present disclosure, there is provided a single step, continuous process for the synthesis of metformin hydrochloride, the process comprising: reacting dimethyl amine hydrochloride and 2-Cyanoguanidine at a temperature in the range of 145-155° C. for a residence time in the range of 300-600 seconds in a vertical screw reactor to obtain crystalline metformin hydrochloride with a purity of at least 95% and conversion of at least 90%.

These and other features, aspects, and advantages of the present subject matter will be better understood with reference to the following description and appended claims. This summary is provided to introduce a selection of concepts in a simplified form. This summary is not intended to identify key features or essential features of the claimed subject matter, nor is it intended to be used to limit the scope of the claimed subject matter.

### BRIEF DESCRIPTION OF THE DRAWINGS

The detailed description is described with reference to the accompanying figures. In the figures, the left-most digit(s) of a reference number identifies the figure in which the reference number first appears. The same numbers are used throughout the drawings to reference like features and components.

FIG. 1 illustrates the screw reactor, in accordance with an implementation of the present disclosure.

FIG. 2 illustrates ¹H-NMR data for the purity of Metformin. HCl, in accordance with an implementation of the present disclosure. FIG. 2 further demonstrates the moisture peak that the product captures from atmosphere during cooling, in accordance with an implementation of the present disclosure.

FIG. 3 corresponds to a product generated by comparative example 1

FIG. 4 corresponds to a product generated by comparative example 2

FIG. 5 corresponds to a product generated by working example 6

FIG. 6 corresponds to a product generated by working example 7

### DETAILED DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

### Definitions

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

Ratios, concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a temperature in the range of 145-155° C. should be interpreted to include not only the explicitly recited limits of about 145° C. to about 155° C. but also to include sub-ranges, such as 146-150° C., and so forth, as well as individual amounts, within the specified ranges, such as 150° C., and 148.9° C.

The terms “white crystalline solid” and “colorless crystalline solid” are used synonymously throughout the specification.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods, and materials are now described. All publications mentioned herein are incorporated herein by reference.

The present disclosure is not to be limited in scope by the specific embodiments described herein, which are intended for the purposes of exemplification only.

Functionally-equivalent products, compositions, and methods are clearly within the scope of the disclosure, as described herein.

To accomplish the objectives of the invention, the inventors disclose a continuous solvent free process for the synthesis of chemicals and pharmaceuticals using a screw reactor. The process as provided by the present disclosure has the advantages of simple and easy operation, high raw material utilization rate, and cheap as well as easily available solvent, the market price of which is 2-3 times lower than prices of solvents in prior art, and the synthesis cost is reduced by about 30%, and thus the preparation method of the invention is very suitable for industrial production of metformin hydrochloride.

The screw reactors as described herein are conventionally used in the polymer industry for synthesis of polymers by melt polymerization and for extrusions. Its application in other domains is uncommon and no such proposals exist in the prior art. Here inventors have envisaged a solvent free process for the synthesis of metformin hydrochloride using a screw reactor. The jacketed single-screw reactor may be constructed from Teflon, glass or any metal selected from. stainless steel SS316, copper, hastelloy and such like for continuous flow reactions involving solids/slurries. Jacket may be made from glass or any polymeric material or metal.

The reactants in powder form are fed using two screw conveyers that help maintain the desired feed rate of individual substrates. The feed is given to another vertically aligned screw with downward flow to avoid any liquid due to shear thinning or melting settling even in the meniscus form or an inclined screw with downward flow direction. It is necessary to mention that having horizontal arrangement of the screw for reaction retains some liquid unless extremely close clearance is kept between the threads and the chamber wall. However, such a situation can create friction in the presence of reaction mass, which can lead to unsafe operations.

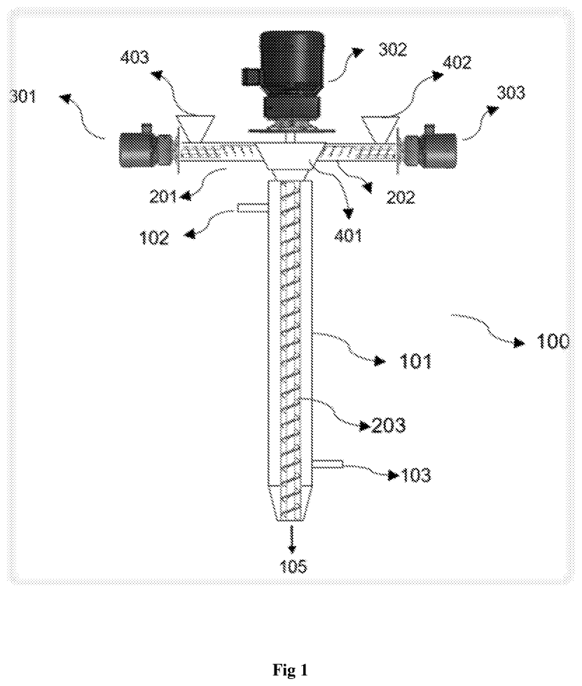

In the present disclosure, vertical alignment for screw reactor (100) (FIG. 1) (having glass or metal jacket) (101) such that the distance between the outer diameter of the screw thread and the inner diameter of the jacket is not more than 0.25 mm is preferred to avoid any liquid to remain accumulated. Any liquid used or generated in the reaction will flow downward due to gravity and any gas generated in the reaction will either flow in the form of packets or escape from the top if the material is not very densely packed. The inlet and outlet ports of the jacket (102 and 103), which may be alternated or swapped, are connected to a constant temperature circulation bath or wrapped with an electric heating tape. For the reactions where reactants are in liquid or in slurry form, pumps were used for dosing instead of screw feeding system. This ensures no accumulation of reactants in the feed section to the reactor.

In the process of the present disclosure, a solid-solid reaction occurs, in the screw reactor. The substrates which are solids, flow and react to form a solid product

Accordingly, in a preferred embodiment, referring to FIG. 1, the screw reactor comprises:

Part (part number)

Jacketed screw reactor (100)
Jacket (101)
Outlet (102) for the heating/cooling fluid from jacket (101)
Inlet (103) for the heating/cooling fluid from jacket (101)
Feed funnel (401, 402, 403) connected to the screw reactor
and to the two reactant dosing sections
Outlet (105) of the screw reactor (100)
Reactant feeding screws (201, 202)
Screw for the reactor (203)
Motors for screw rotation (301, 302, 303)

In an embodiment of the present disclosure, there is provided a single step, continuous process for the synthesis of metformin hydrochloride, the process comprising: reacting dimethyl amine hydrochloride and 2-Cyanoguanidine at a temperature in the range of 145-155° C. for a residence time in the range of 300-600 seconds in a vertical screw reactor to obtain crystalline metformin hydrochloride with a purity of at least 95% and conversion of at least 90%.

In an embodiment of the present disclosure, there is provided a single step, continuous process for the synthesis of metformin hydrochloride, the process comprising: reacting dimethyl amine hydrochloride and 2-Cyanoguanidine at a temperature in the range of 145-155° C. for a residence time in the range of 300-600 seconds in a vertical screw reactor to obtain crystalline metformin hydrochloride with a purity of at least 95% and conversion of at least 90%, wherein said dimethyl amine hydrochloride and 2-Cyanoguanidine is in 1:1 equivalent ratio.

In another embodiment of the present disclosure, there is provided a single step, continuous process for the synthesis of metformin hydrochloride, the process consisting: reacting dimethyl amine hydrochloride and 2-Cyanoguanidine at a temperature in the range of 145-155° C. for a residence time in the range of 300-600 seconds in a vertical screw reactor to obtain crystalline metformin hydrochloride with a purity of at least 95% and conversion of at least 90%.

In an embodiment of the present disclosure, there is provided a single step, continuous process for the synthesis of metformin hydrochloride, the process comprising: reacting dimethyl amine hydrochloride and 2-Cyanoguanidine at a temperature in the range of 145-150° C. for a residence time in the range of 300-600 seconds in a vertical screw reactor to obtain crystalline metformin hydrochloride with a purity of at least 95% and conversion of at least 90%.

In an embodiment of the present disclosure, there is provided a single step, continuous process for the synthesis of metformin hydrochloride, the process comprising: reacting dimethyl amine hydrochloride and 2-Cyanoguanidine at a temperature of 150° C. for a residence time in the range of 300-600 seconds in a vertical screw reactor to obtain crystalline metformin hydrochloride with a purity of at least 95% and conversion of at least 90%.

In yet another embodiment of the present disclosure, there is provided a single step, continuous process for the synthesis of metformin hydrochloride, the process comprising: reacting dimethyl amine hydrochloride and 2-Cyanoguanidine at a temperature in the range of 145-155° C. for a residence time in the range of 300-600 seconds in a vertical screw reactor to obtain crystalline metformin hydrochloride with a purity of more than 95% and conversion of at least 90%. In another embodiment of the present disclosure, there is provided a process as described herein, wherein said metformin hydrochloride has purity of more than 97%. In an embodiment of the present disclosure, there is provided a process as described herein wherein said metformin hydrochloride has purity of more than 98%.

In yet another embodiment of the present disclosure, there is provided a single step, continuous process for the synthesis of metformin hydrochloride, the process comprising: reacting dimethyl amine hydrochloride and 2-Cyanoguanidine at a temperature in the range of 145-155° C. for a residence time in the range of 300-600 seconds in a vertical screw reactor to obtain crystalline metformin hydrochloride with a purity of at least 95% and conversion of at least 90%, wherein said process results in more than 95% selectivity towards metformin hydrochloride. In another embodiment of the present disclosure, there is provided a process as described herein, wherein said process results in more than 98% selectivity towards metformin hydrochloride. In an embodiment of the present disclosure, there is provided a process as described herein, wherein said process results in 100% selectivity towards metformin hydrochloride.

In yet another embodiment of the present disclosure, there is provided a single step, continuous process for the synthesis of metformin hydrochloride, the process comprising: reacting dimethyl amine hydrochloride and 2-Cyanoguanidine at a temperature in the range of 145-155° C. for a residence time in the range of 300-600 seconds in a vertical screw reactor to obtain crystalline metformin hydrochloride with a purity of at least 95% and conversion of at least 95%. In an embodiment of the present disclosure, there is provided a process as described herein, wherein said conversion is 100%.

In a further embodiment of the present disclosure, there is provided a single step, continuous process for the synthesis of metformin hydrochloride, the process comprising: reacting dimethyl amine hydrochloride and 2-Cyanoguanidine at a temperature in the range of 145-155° C. for a residence time in the range of 300-600 seconds in a vertical screw reactor to obtain crystalline metformin hydrochloride with a purity of at least 95% and conversion of at least 90%, wherein said dimethyl amine hydrochloride and 2-Cyanoguanidine are in solid form. In another embodiment of the present disclosure, there is provided a process as described herein, wherein said dimethyl amine hydrochloride and 2-Cyanoguanidine are free flowing dry powders.

In an embodiment of the present disclosure, there is provided a continuous solvent free process for the synthesis of metformin hydrochloride comprises of reacting a 1:1 equivalent of dimethyl amine hydrochloride and 2-Cyanoguanidine at 150° C. for a period ranging from 300-600 seconds in a vertical screw reactor operated at 95 rpm to obtain white, crystalline metformin hydrochloride of 95% purity, wherein conversion of the reactants is ranging from 97-100%, selectivity towards metformin hydrochloride is 95%, and wherein the process is a reaction between solid dimethyl amine hydrochloride and solid 2-Cyanoguanidine; wherein said dimethyl amine hydrochloride and 2-Cyanoguanidine contain 0% moisture and are free of agglomerates.

In an embodiment of the present disclosure, there is provided a single step, continuous process for the synthesis of metformin hydrochloride, the process comprising: reacting dimethyl amine hydrochloride and 2-Cyanoguanidine at a temperature in the range of 145-150° C. for a residence time in the range of 300-600 seconds in a vertical screw reactor to obtain crystalline metformin hydrochloride with a purity of at least 95% and conversion of at least 90%, wherein said process is solvent-free.

In yet another embodiment of the present disclosure, there is provided a single step, continuous process for the synthesis of metformin hydrochloride, the process comprising: reacting dimethyl amine hydrochloride and 2-Cyanoguanidine at a temperature in the range of 145-150° C. for a residence time in the range of 300-600 seconds in a vertical screw reactor to obtain crystalline metformin hydrochloride with a purity of at least 95% and conversion of at least 90%, wherein said process is free of additives.

In another embodiment of the present disclosure, there is provided a process as described herein, wherein said dimethyl amine hydrochloride and 2-Cyanoguanidine undergo atleast 90% conversion, and provide greater than 90% selectivity to desired product. The process of the present disclosure avoids formation of colored by-products or impurities in the desired products.

In a preferred embodiment, the process of the present disclosure results in the synthesis of Metformin HCl with greater than 90% conversion of substrates employed and >90% selectivity towards metformin.

Although the subject matter has been described with reference to specific embodiments, this description is not meant to be construed in a limiting sense. Various modifications of the disclosed embodiments, as well as alternate embodiments of the subject matter, will become apparent to persons skilled in the art upon reference to the description of the subject matter. It is therefore contemplated that such modifications can be made without departing from the spirit or scope of the present subject matter as defined.

The invention will now be explained with specific exemplary embodiment, which should not be construed as limiting the scope of the invention in any manner. It is quite non obvious for a person involved in synthetic chemistry to anticipate the use of screw reactor/conveyor for routine chemical synthesis in a continuous manner, more particularly, a solvent fee synthetic approach for high temperature exothermic reaction.

### EXAMPLES

The disclosure will now be illustrated with the working examples, which is intended to illustrate the working of disclosure and not intended to take restrictively to imply any limitations on the scope of the present disclosure. Unless defined otherwise, all technical and scientific terms used herein have the same meaning as commonly understood to one ordinary person skilled in the art to which this disclosure belongs. Although methods and materials similar or equivalent to those described herein can be used in the practice of the disclosed methods and compositions, the exemplary methods, devices and materials are described herein. It is to be understood that this disclosure is not limited to particular methods, and experimental conditions described, as such methods and conditions may apply.

### General Process for Preparation of Metformin Hydrochloride:

In a comparative embodiment, in a continuous flow synthesis of Metformin hydrochloride, solid dimethyl amine hydrochloride (1 equivalent) and solid 2-Cyanoguanidine (1 equivalent) were continuously fed into the vertical screw reactor (100) (Specifications: SS316, I.D=1.27 cm, length=30 cm, screw I.D=1.0 cm, length=29 cm) at 150° C. with residence time of 6 sec. The process resulted in the generation of hydrochloric acid at elevated temperature of the reaction, and the reaction mass turned black in color, and could not be characterized.

In another comparative embodiment, reaction temperature of greater than 150° C. provide a coloured product, and a residence time of less than 100 seconds resulted in poor conversions of substrates.

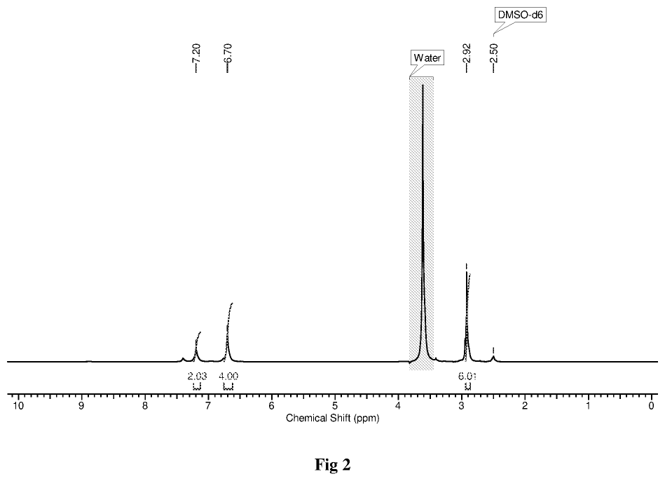

In the experimental embodiment, the solid dimethyl amine hydrochloride (1 equivalent) and solid 2-Cyanoguanidine (1 equivalent) were continuously fed into the jacketed vertical PTFE screw reactor (100) at a temperature ranging from 100-150° C. for 100-600 seconds at rpm in the range of 50-500 to obtain the desired solid product, with at least 90% selectivity towards metformin. The conversion of the substrates is atleast 90%. The product formed is metformin hydrochloride and is characterized by the formation of white crystalline product, with no trace of impurities (FIG. 2).

### COMPARATIVE EXAMPLES AND WORKING EXAMPLES

### Comparative Example 1

In continuous flow synthesis of Metformin hydrochloride, solid dimethyl amine hydrochloride (1 equivalent) and solid 2-Cyanoguanidine (1 equivalent) were continuously fed into the vertical screw reactor (I.D=1.27 cm, length=30 cm, screw I.D=1.0 cm, length=29 cm) at 150° C. with residence time of 6 sec. Generation of hydrochloric acid at elevated temperature the reaction mass turned black in color mass (FIG. 3).

### Comparative Example 2

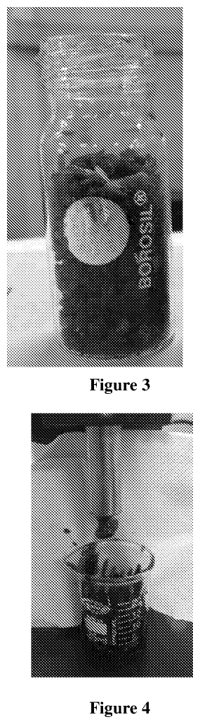

For the experimental set-up as given in Comparative Example 1, at 160° C. at residence time of 6 s, again generation of hydrochloric acid at elevated temperature the reaction mass turned black in color mass with no desired product (FIG. 4).

### Comparative Example 3

For the experimental set-up as given in Comparative Example 1, at 120° C. and with residence time of 6 s, colourless reaction mass was collected having 18% conversion of the substrates.

### Comparative Example 4

For the experimental set-up as given in Comparative Example 3, at identical conditions and set-up, upon increasing the residence time to 48 s, colourless semi-solid mass with 50% conversion and 100% selectivity of metformin was obtained.

### Working Example 5

For the experimental set-up as given in Comparative Example 3, in the same set-up at 150° C., upon increasing the residence time to 100 s, colourless semi-solid mass with 90% conversion and 95% selectivity of metformin was obtained.

### Working Example 6

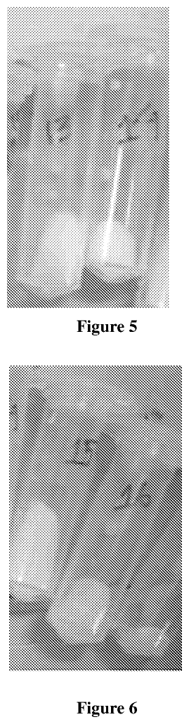

At the experimental conditions and in the experimental set-up as given in Comparative Example 3, at 150° C., upon increasing the residence time to 300 s, colourless semi-solid mass with 97% conversion and 95% selectivity of metformin was obtained. Semi solid is the form when it exits from the reactor before it crystallizes on its own at 25-30° C. during collection (FIG. 5).

### Working Example 7

At the experimental conditions and in the experimental set-up as given in Comparative Example 3, at 150° C., upon increasing the residence time to 600 s, colourless semi-solid mass with 100% conversion and 95% selectivity of metformin was obtained. Semi solid is the form when it exits from the reactor before it crystallizes on its own at 25-30° C. during collection (FIG. 6).

The examples explained above proved that the metformin hydrochloride with higher purity and yield was achieved only by the process disclosed in the present disclosure. Any deviation in the process parameters such as rotating or screw speed, temperature, residence time resulted in the metformin hydrochloride with poor characteristics, in terms of yield, purity, conversion rate. Hence, the present disclosure provides a process for producing metformin hydrochloride which is economically viable.

### Advantages of the Invention

The present disclosure provides a process for the production of metformin hydrochloride, including in industrial scale. The process of the present disclosure avoids the use of solvent and additional additives, which in turn makes the process economically suitable. The crystalline metformin hydrochloride obtained from the process of the present disclosure is highly pure which may be directly used for further processing without additional step of purification. The process also involves the use of stoichiometric quantities. The process of the present disclosure includes other economic advantages, in terms of cost.

1. A single step, continuous process for the synthesis of metformin hydrochloride, the process comprising:
reacting dimethyl amine hydrochloride and 2-Cyanoguanidine at a temperature in the range of 145-155° C. for a residence time in the range of 300-600 seconds in a vertical screw reactor to obtain crystalline metformin hydrochloride with a purity of at least 95% and conversion of at least 90%.

2. The process as claimed in claim 1, wherein said dimethyl amine hydrochloride and 2-Cyanoguanidine is in 1:1 equivalent ratio.

3. The process as claimed in claim 1, wherein said process results in more than 95% selectivity towards metformin hydrochloride.

4. The process as claimed in claim 1, wherein said dimethyl amine hydrochloride and 2-Cyanoguanidine are in solid form.

5. The process as claimed in claim 1, wherein said process is solvent-free.

6. The process as claimed in claim 1, wherein said process is free of additives.

7. The process as claimed in claim 4, wherein said dimethyl amine hydrochloride and 2-Cyanoguanidine are free flowing dry powders.
